(19) United States
(12) Patent Application Publication
(10) Pub. No.: US 2017/0281640 A1
(43) Pub. Date: Oct. 5, 2017

(54) Pharmaceutical Compositions Containing a Muscle Relaxant and a Nonsteroidal Anti-Inflammatory Drugs (NSAID)

(71) Applicant: Productos Maver, S.A. de C.V., Tlaquepaque (MX)

(72) Inventors: Silvia Romero Medina, Tlaquepaque (MX); Jorge Castro Luna, Tlaquepaque (MX); Karim Saul Solorza Camacho, Tlaquepaque (MX); Alejandro Maldonado Pérez, Tlaquepaque (MX)

(21) Appl. No.: 15/089,980

(22) Filed: Apr. 4, 2016

(51) Int. Cl. A61K 31/5415; A61K 9/48; A61K 31/325; A61K 9/16

(52) U.S. Cl. CPC A61K 31/5415; A61K 9/1617; A61K 9/1652; A61K 9/1635; A61K 9/1623; A61K 9/4808; A61K 9/1682; A61K 31/325; A61K 9/4858; A61K 9/4866

(57) ABSTRACT

Provided herein is a stable pharmaceutical composition including meloxicam and methocarbamol in the form of granules, including a meglumine and sodium citrate complex to improve solubility. The pharmaceutical compositions are administered simultaneously and are kept separated in the pharmaceutical form that is administered. Also provided herein are methods for the treatment of disorders associated with muscle, skeletal muscle, and pain.

### FIELD OF THE INVENTION

This invention refers to stable pharmaceutical compositions for the treatment of disorders associated with muscle, skeletal muscle and pain related problems. Such compositions comprise a granular material containing a muscle relaxant drug and a selected drug of the NSAID group. The drugs of the invention are in a separated pharmaceutical form, but they are simultaneously administered.

### BACKGROUND OF THE INVENTION

Meloxicam or 4-hydroxy-2-methyl-N-(5-methyl-2-thiazolil)-2H-1,2-benzothiazine-3-carboxamide 1,1-dioxide is a nonsteroidal anti-inflammatory drug indicated for the treatment of rheumatoid arthritis, osteoarthritis, spondylitis, gouty arthritis, non-rheumatoid inflammatory ailments and upper airways acute nonbacterial inflammatory processes.

Meloxicam, meglumine and sodium salts, as well as a preparation process thereof, are described in U.S. Pat. No. 4,233,299.

Meloxicam is a non-readily dissolvable drug, however, meloxicam salts, and meglumine salt in particular, show an improved solubility in function of the pH. The preparation processes of the meloxicam salts are disclosed in U.S. Pat. Nos. 6,869,948 and 8,920,820.

Methocarbamol or 3-(2-methoxyphenxy)-1,2-propanediol 1-carbamate is a muscle relaxant compound that is considered as an adjuvant in acute painful musculoskeletal disorders.

Methocarbamol and its manufacturing processes have been described in U.S. Pat. No. 2,770,649.

It is known in the art that the combination of a nonsteroidal anti-inflammatory compound and a muscle relaxant compound produces higher analgesic and antinflammatory effects, mainly in the treatment of musculoskeletal disorders and related disorders, where pain, inflammation, muscle spasm, and limited mobility such as trauma and muscle strains, back pain, stiff neck, bursitis, arthritis, tendinitis and muscle contractures coexist.

Nowadays, there are products containing the meloxicam and methocarbamol combination in a capsule form, for oral administration for the treatment of musculoskeletal conditions, known commercially under the name of DOLOCAM PLUS®. In Mexico, such product is marketed by Representaciones e Investigaciones Medicas S.A. de C. V.

Several different compositions containing the combination of meloxicam and methocarbamol have been described in the art.

Mexican Patent 249,290, discloses a composition in a capsule form, which comprises Meloxicam at a concentration of 0.4 and 20%, Methocarbamol at a concentration of 20 and 80%, magnesium hydroxide, lactose, sodium glycolate starch, magnesium stearate, and other excipients.

Mexican Patent 268,712 discloses a composition in the form of coated microspheres that comprise a) inert cores coated with a first film containing a muscle relaxant such as meloxicam of modified release; b) a second retardant polymeric film; c) a third film containing an NSAID such as methocarbamol of immediate release.

Therefore, in the pharmaceutical field, there is a need for solid pharmaceutical compositions for oral administration containing meloxicam and methocarbamol of greater stability and improved dissolution.

### SUMMARY OF THE INVENTION

Therefore, it is an object of the present invention to provide stable pharmaceutical compositions containing a muscle relaxant drug such as meloxicam, and a selected drug from the NSAID group, such as methocarbamol, in the form of granulated material. The drugs of the present invention are administered simultaneously keeping them in separated forms in the administered pharmaceutical form. Such compositions are useful for the treatment of disorders associated with muscle, skeletal muscle and from mild to severe pain related problems.

### BRIEF DESCRIPTION OF THE DRAWINGS

FIG. 1 shows the dissolution profile at pH 1.2, reference product versus formulation of the present invention.

FIG. 2 illustrates the dissolution profile at pH 4.5, reference product versus formulation of the invention.

FIG. 3 shows the dissolution profile at pH 6.8, reference product versus formulation of the invention.

FIG. 4 illustrates the plasmatic concentration for formulation 1, batches A and B, meloxicam drug.

FIG. 5 shows the plasmatic concentration for formulation 1, batches A and B, methocarbamol drug.

### DETAILED DESCRIPTION OF THE INVENTION

This invention describes a pharmaceutical composition useful for the treatment of disorders related to muscle, skeletal muscle, problems related to rheumatoid arthritis, osteoarthritis, spondylitis, gouty arthritis, acute and chronic inflammatory conditions nonrheumatic, nonbacterial acute inflammatory processes of upper airways and moderate to severe pain and its manufacturing process.

The described pharmaceutical composition is a formulation for oral administration that contains a muscle relaxant drug and a selected drug of the NSAIDs group in the form of granules contained in a capsule for oral administration, where the release of the drugs is immediate.

The present invention describes a formulation that contains the combination of the methocarbamol drugs and/or pharmaceutically acceptable salts, pro-drugs, derivatives, polymorphic crystals, amorphic, metabolites and/or combination of the same; and meloxicam and/or its pharmaceutically acceptable salts, pro-drugs, derivatives, polymorphic crystals, amorphic, metabolites and/or combination of the same in the form of granules that keep the drugs separated.

Granulate number 1 consists of a support created from meloxicam; meglumine, sodium citrate; a moisturizing solution with a 1:1 mixture of ethanol and water; at least a surfactant, such surfactant is selected in a non-limiting manner from sodium docusate, glyceryl monooleate, phospholipids, alpha tocopherol, cetrimide, docusate sodium, polyoxylglycerides, potassium sorbate, sorbic acid, sodium lauryl sulfate; a diluent agent, such agent is selected in a non-limiting manner from starch, pregelatinized maize starch, corn starch, sugar, compressible sugar, calcium carboxymethylcellulose, cellulose, microcrystalline cellulose, ethylcellulose, hydroxyethyl cellulose, hydroxymethyl cellulose, hydroxypropyl cellulose, hydroxypropylmethyl cellulose, hydroxypropyl starch, methyl cellulose, lactose monohydrate, sorbitol, sucrose, talc tragacanth, trehalose, xylitol and combinations of the same; a disintegrant agent, such disintegrant agent is selected in a non-limiting manner from alginic acid, crospovidone, ion exchange resins, aluminum silicate, magnesium silicate, microcrystalline cellulose, starch, sodium starch glycolate, modified cellulose gum, PVP, dodecyl sulfate sodium corn starch, carboxymethylcellulose calcium, rice starch, crosslinked N-vinyl-2-pyrrolidone, sodium croscarmellose, formaldehyde-casein and combinations of the same; and a binder, such binder is selected in a non-limiting manner from alginic acid, sodium alginate, starch, pregelatinized starch, calcium carbonate, carbomer, calcium carboxymethylcellulose, cellulose, microcrystalline cellulose, copovidone, dextrates, dextrin, dextrose, ethylcellulose, gelatin, guar gum, hydroxyethyl cellulose, hydroxymethyl cellulose, hydroxypropyl low-substitution hydroxypropyl starch, methyl cellulose, inulin, calcium lactate, lactose monohydrate, aluminum silicate and magnesium, maltodextrin, methylcellulose, polycarbophil, polydextrose, chitosan cellulose, polyethylene oxide, polymethacrylates, povidone, polyvidone, polyethylene glycol succinate or sucrose and combinations of the same.

In a modality of the present invention, the granulated material number 1 contains, at least in an optional way, an additional alkalinizing agent selected from sodium hydroxide, calcium hydroxide, potassium hydroxide, diethanolamine, potassium bicarbonate, sodium bicarbonate and potassium citrate.

The granulated material number 2 consists of a support created from methocarbamol; a moisturizing solution with a mix 1:1 of ethanol and water; and at least one agglutinant agent, such agglutinant agent is selected in a non-limiting manner from alginic acid, sodium alginate, starch, pregelatinized starch, calcium carbonate, carbomer, calcium carboxymethylcellulose, cellulose, microcrystalline cellulose, copovidone, dextrates, dextrin, dextrose, ethylcellulose, gelatin, guar gum, hydroxyethyl cellulose, hydroxymethyl cellulose, hydroxypropyl cellulose of low-substitution, hydroxypropyl starch, methyl cellulose, inulin, calcium lactate, lactose monohydrate, aluminum silicate and magnesium, maltodextrin, methylcellulose, polycarbophil, polydextrose, chitosan, polyethylene oxide, polymethacrylates, povidone, polyvidone, sucrose and succinate polyethylene glycol or combinations of the same.

Optionally, a lubricant is included in one and/or both granulated materials, such lubricating agent is selected in a non-limiting manner from calcium stearate, magnesium stearate, stearic acid, zinc stearate, hydrogenated castor oil, glyceryl behenate, glyceryl monostearate, mineral oil, polyethylene glycol, sodium stearyl fumarate, sodium benzoate and talc.

The invention describes the process for obtaining the formulation that contains the meloxicam drug and methocarbamol, where such process is characterized in that the two granulated materials are prepared separately.

The described composition in this invention is characterized as containing a granulated material number 1 that contains meloxicam and/or its pharmaceutically acceptable salts, pro-drugs, derivatives, polymorphic crystals, amorphic, metabolites and/or combinations of the same in an effective therapeutic quantity; meglumine in a proportion of 0.5% to 10% of weight in the formulation; sodium citrate in a proportion of 12% to 22% of weight in the formulation; at least a surfactant agent in a proportion from 3% to 15% of weight in the formulation; at least a diluent agent in a proportion from 40% to 80% of weight in the formulation; at least a disintegrant agent in a proportion from 1% to 10% of weight in the formulation; at least an agglutinant agent in a proportion from 0.5% to 10% of weight in the formulation; a moisturizing solution with a mixture of ethanol and water in a necessary proportion such as to obtain a granulated material.

The described composition in this invention is characterized as containing a granulated material number 2 that contains methocarbamol and/or its pharmaceutically acceptable salts, pro-drugs, derivatives, polymorphic crystals, amorphic, metabolites and/or combinations of the same in a pharmaceutically effective quantity; at least an agglutinant agent in a proportion from 0.5% to 10% of weight in the formulation; and a moisturizing solution with a mixture of ethanol and water in a necessary proportion such as to obtain a granulated material.

The process for obtaining granulated material number 1 is characterized by the formation of the granulation solution, where meglumine and a mixture of ethanol and water are combined until a homogenization point, in which the mixture of ethanol and water is prepared in a proportion 1:1, and later meloxicam is added. This stage of the process is very important, since in this stage solubility increases, and consequently, meloxicam bioavailability also increases from the interaction of sodium meglumine-citrate with the drug.

Later, the granulation support is manufactured by making a mixture of sodium citrate with a tensioactive agent, a diluent agent, a disintegrant agent, and an agglutinant agent. After that, the support is moisturized with the granulation solution until granulation point, the wet granule obtained is sieved with a mesh with a pore size from 1.0 mm to 8.00 and it is dried at a temperature from 20° C. to 60° C. When the granule is dry, it is sieved with a mesh with a pore size from 0.2 mm to 1.5 mm. The process is characterized in that in the drying stage the granule loses no more than 5% of its total weight, obtaining a granulated material that presents a uniformity of content of no less than 90% and no more than 110% of the meloxicam content.

The process for obtaining granulated material 2 is characterized by the formation of the granulation solution, where a mixture of ethanol and water is prepared until the homogenization point, in which the mixture of ethanol and water is prepared in 1:1 proportion. Later, the granulation support where methocarbamol and an agglutinant agent are mixed is manufactured. Afterwards, the support is moisturized with the granulation solution until the granulation point, the wet granule obtained is sieved with a mesh with a pore size of 1.0 mm to 8.0 mm and it is dried at a temperature between 20° C. to 60° C. When the granule is dry, it is sieved with a mesh of a pore size of 0.2 mm to 1.5 mm. The process is characterized in that the granule does not lose more than 5% of its total weight in the drying stage, obtaining a granulated material that presents the content of no less than 90% and no more than 110% of methocarbamol content.

In order to obtain the final composition, granulated materials 1 and 2 are mixed for a period of 4 to 20 minutes or until a homogenous mixture is obtained, optionally to the mixture a lubricant agent is added and mixed for a period of 4 to 20 minutes or until a homogenous mixture is obtained. The final mixture is formulated for oral administration in a capsule.

Such pharmaceutical form is characterized by having a dissolution with a percentage of dissolution of the drug versus time, of Q≧60% for meloxicam, and Q≧85% for methocarbamol. In which the obtained composition presents a meloxicam and methocarbamol content of no less than 90% and no more than 110%.

The described formulations in the present invention provide a uniformity of dosage, the same that were determined by the mass-variation method, in which, 10 units were weighed separately, their content was emptied and their net weight was obtained, content was assessed to obtain the value of the drug for each unit. This procedure was performed for a batch A and a batch B, and the drugs meloxicam and methocarbamol were evaluated.

The obtained results for the meloxicam in the batch A had a mean of 98.9% with a standard deviation of 3.1 (table 1), for batch B the obtained mean was 99.9% and a standard deviation of 4.2% (table 2), for methocarbamol, batch A presents a mean of 100% and a standard deviation of 2.9% (table 3) and for batch B a mean of 100.99% was obtained with a standard deviation of 3.8% (table 4)

TABLE 1
Assessment of dosage uniformity, batch A. Meloxicam drug
Batch A meloxicam

| Unit | 1 | 2 | 3 | 4 | 5 | 6 | 7 | 8 | 9 | 10 |
|---|---|---|---|---|---|---|---|---|---|---|
| Assessment % | 98.89 | 98.97 | 98.89 | 98.92 | 98.89 | 98.86 | 98.89 | 98.92 | 98.89 | 98.86 |

TABLE 2
Assessment of dosage uniformity, batch B. Meloxicam drug
Batch B meloxicam

| Unit | 1 | 2 | 3 | 4 | 5 | 6 | 7 | 8 | 9 | 10 |
|---|---|---|---|---|---|---|---|---|---|---|
| Assessment % | 99.90 | 99.98 | 99.90 | 99.93 | 99.90 | 99.87 | 99.82 | 99.93 | 99.90 | 99.87 |

TABLE 3
Assessment of dosage uniformity, batch A. Active methocarbamol
Batch A methocarbamol

| Unit | 1 | 2 | 3 | 4 | 5 | 6 | 7 | 8 | 9 | 10 |
|---|---|---|---|---|---|---|---|---|---|---|
| Assessment % | 99.99 | 100.07 | 99.99 | 100.2 | 99.99 | 99.99 | 99.99 | 100.2 | 99.99 | 99.96 |

TABLE 4
Assessment of dosage uniformity, batch B. Active methocarbamol
Batch B methocarbamol

| Unit | 1 | 2 | 3 | 4 | 5 | 6 | 7 | 8 | 9 | 10 |
|---|---|---|---|---|---|---|---|---|---|---|
| Assessment % | 101.00 | 101.00 | 101.00 | 100.95 | 101.00 | 101.09 | 101.00 | 101.98 | 100.95 | 100.98 |

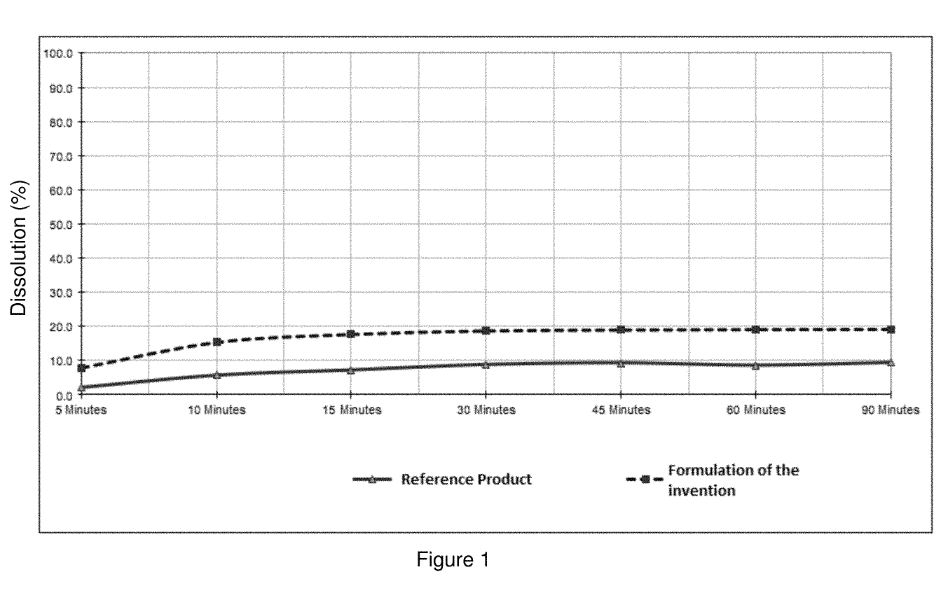

The dissolution of the formulation of this invention was evaluated and compared with respect to the reference product, such dissolution was carried out in dissolution media with phosphate buffer solutions and pH of 1.2, 4.5 and 6.8. Meloxicam and methocarbamol drugs were assessed. In the test with pH 1.2 (FIG. 1), it is observed with better clarity the better dissolution of the meloxicam by the formulation of the present invention in comparison to the reference product.

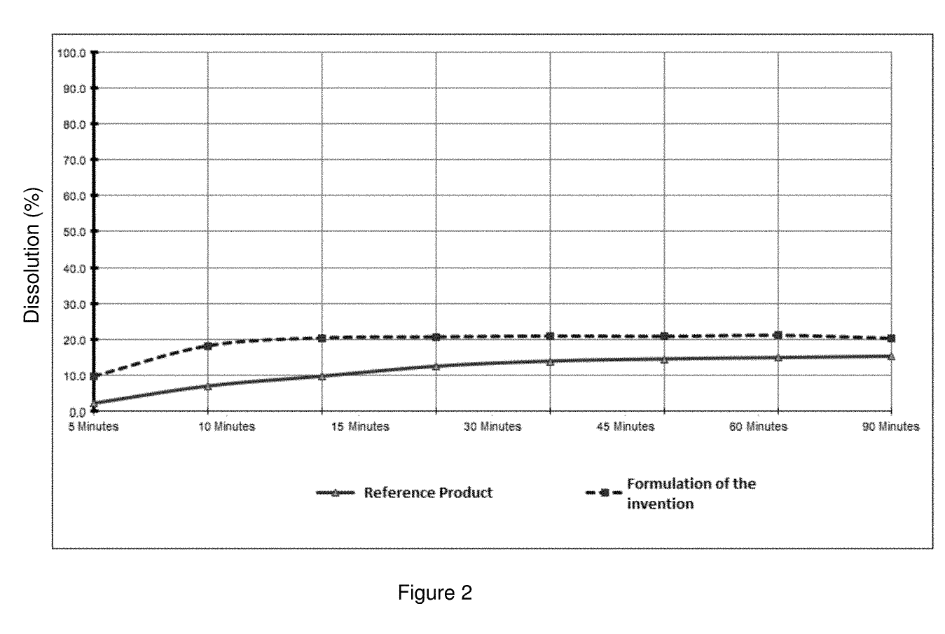

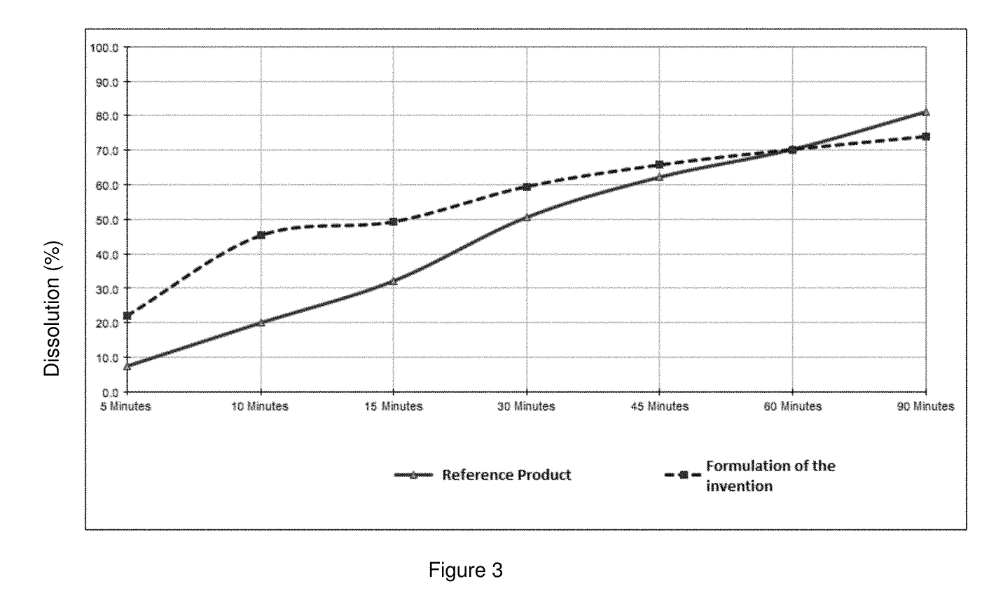

This phenomenon is observed in the dissolutions performed at pH 4.5 (FIG. 2) 6.8 (FIG. 3). This evaluation shows that the meloxicam-meglumine-sodium citrate complex considerably improves the dissolution of the meloxicam drug.

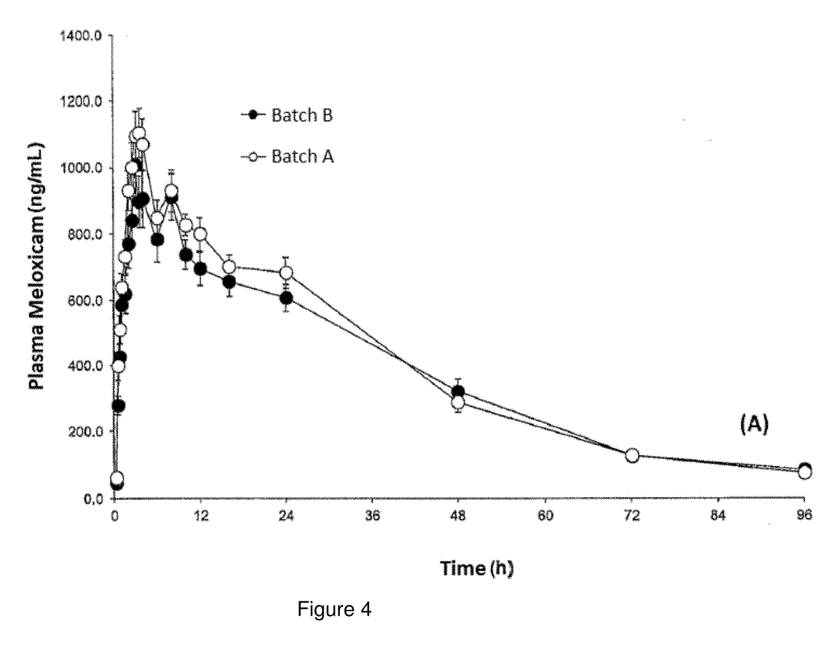

Additionally, a clinic study was developed for two batches (A and B) of the described formulations in examples 1 and 7 in this invention. The study was developed with 27 volunteers with an average age of 28.58 years, with a size of 1.66 meters, a weight of 64.77 kilograms and a BMI of 23.27% Kg/m². The obtained results demonstrate that there is sufficient statistical evidence to conclude that both batches of the formulation of example 1 present a maximum plasma concentration (Cmax) of meloxicam of 1095.90 ng/mL for Batch A and 1008.94 ng/mL for Batch B and a tmax of 2.520 hours and 2.489 hours, respectively, after their oral intake of meloxicam drug (FIG. 4).

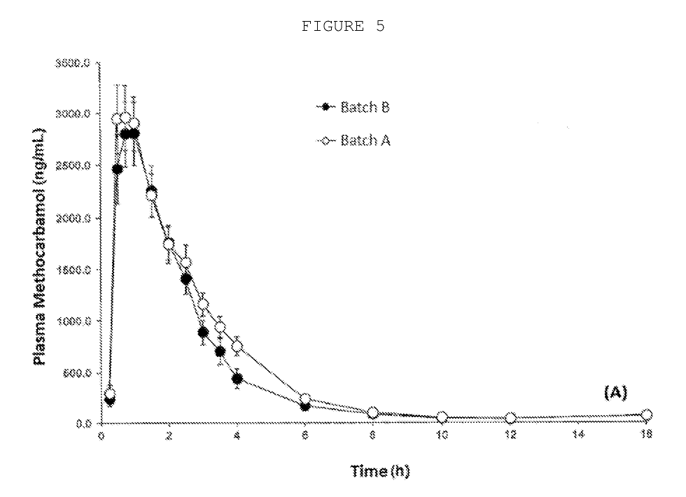

Methocarbamol showed a maximum plasma concentration (Cmax) of 2900.52 ng/mL for batch A and 2804.41 ng/mL for batch B and Tmax of 0.79 hours and 0.76 hours, respectively, after oral intake of the methocarbamol drug (FIG. 5).

The composition of the present invention presents high stability in both the pharmaceutical form and the active ingredients, which was demonstrated in an accelerated stability study of the composition by performing the corresponding analysis of appearance, loss by drying, degradation compounds, dissolution of both drugs, and microbial limits. Assessment of the drugs at times 0, 3, 6, 9, 12, 18 and 24 months, with storing conditions of 30° C.±2° C. with a 65%±5% of relative humidity was conducted. The results obtained at the conclusion of the study demonstrated that the composition was stable, keeping the quantity of meloxicam and methocarbamol, evaluated via the assessment, fulfilling the specifications of drying loss of no more than 5%, a dissolution of Q≧60% with respect to meloxicam and Q≧85% with respect to methocarbamol and an assessment of no less than 90.0% and no more than 110.0%, the degradation compounds were not higher than 0.5% for each drug and were not higher than 1.0% in total.

### EXAMPLES

Different compositions are presented in a descriptive, non-limiting manner.

### Example 1: Composition of Granule 1 of Meloxicam

| Component | Quantity (%) |
|---|---|
| Meloxicam | 11.20 |
| Sodium lauryl sulfate | 6.00 |
| Sodium citrate | 17.00 |
| Meglumine | 2.00 |
| Pregelatinized starch | 56.50 |
| Crospovidone | 4.00 |
| Polividone | 3.30 |
| Alcohol | c.b.p. |
| Purified water | c.b.p. |

### Example 2: Composition of Granule 1 of Meloxicam

| Component | Quantity (%) |
|---|---|
| Meloxicam | 13.50 |
| Sodium lauryl sulfate | 2.50 |
| Sodium citrate | 18.00 |
| Meglumine | 0.50 |
| Pregelatinized starch | 59.00 |
| Crospovidone | 2.0 |
| Polividone | 4.50 |
| Alcohol | c.b.p. |
| Purified water | c.b.p. |

### Example 3: Composition of Granule 1 of Meloxicam

| Component | Quantity (%) |
|---|---|
| Meloxicam | 5.9 |
| Sodium lauryl sulfate | 6.0 |
| Sodium hydroxide | 18.0 |
| Meglumine | 3.0 |
| Starch | 60.0 |
| PVP | 4.0 |
| Povidone | 3.0 |
| Alcohol | c.b.p. |
| Purified water | c.b.p. |

### Example 4: Composition of Granule 1 of Meloxicam

| Component | Quantity (%) |
|---|---|
| Meloxicam | 7.00 |
| Sodium docusate | 5.50 |
| Sodium hydroxide | 12.50 |
| Meglumine | 4.50 |
| Hydroxipropyl cellulose | 64.50 |
| PVP | 2.70 |
| Povidone | 3.30 |
| Alcohol | c.b.p. |
| Purified water | c.b.p. |

### Example 5: Composition of Granule 1 of Meloxicam

| Component | Quantity (%) |
|---|---|
| Meloxicam | 4.7 |
| Sodium lauryl sulfate | 9.0 |
| Sodium citrate | 22.0 |
| Meglumine | 5.0 |
| Starch | 49.30 |
| PVP | 6.50 |
| Polividone | 3.50 |
| Alcohol | c.b.p. |
| Purified water | c.b.p. |

### Example 6: Composition of Granule 1 of Meloxicam

| Component | Quantity (%) |
|---|---|
| Meloxicam | 15.50 |
| Sodium lauryl sulfate | 6.50 |
| Sodium citrate | 18.0 |
| Meglumine | 4.50 |
| Pregelatinized starch | 47.0 |
| PVP | 5.50 |
| Povidone | 3.0 |
| Alcohol | c.b.p. |
| Purified water | c.b.p. |

### Example 7: Composition of Granule 2 of Methocarbamol

| Component | Quantity (%) |
|---|---|
| Methocarbamol | 93.10 |
| Polividone | 6.90 |
| Alcohol | c.b.p. |
| Purified water | c.b.p. |

### Example 8: Composition of Granule 2 of Methocarbamol

| Component | Quantity (%) |
|---|---|
| Methocarbamol | 88.50 |
| Povidone | 11.5 |
| Alcohol | c.b.p. |
| Purified water | c.b.p. |

### Example 9: Composition of Granule 2 of Methocarbamol

| Component | Quantity (%) |
|---|---|
| Methocarbamol | 80.50 |
| Lactose monohydrate | 19.5 |
| Alcohol | c.b.p. |
| Purified water | c.b.p. |

1. An oral pharmaceutical composition comprising:
i. a first granulated material containing meloxicam, meglumine, sodium citrate, a surfactant agent, a diluent agent, a disintegrant agent, an agglutinant agent, and a moisturizing solution with a mixture of a pharmaceutically acceptable alcohol and water;
ii. a second granulated material containing methocarbamol or its pharmaceutically acceptable salts, an agglutinant agent, and a moisturizing solution with a mixture of ethanol and water; and
iii. optionally, a lubricant agent,
wherein the proportion of sodium citrate to meglumine is in the range of 8:1 to 4:1.

2. The pharmaceutical composition of claim 1, wherein the methocarbamol and meloxicam drugs are not in the same granule.

3. The pharmaceutical composition of claim 1, wherein the first and second granulated materials are contained in a capsule.

4. The pharmaceutical composition of claim 1, wherein the first granulated material is obtained by
preparing a first granulation solution comprising meglumine and a mixture of ethanol and water;
adding meloxicam to the first granulation solution;
manufacturing a first granulation support comprising a mixture of sodium citrate with a tensioactive agent, a diluent agent, a disintegrant agent, and an agglutinant agent;
moisturizing the first granulation support with the first granulation solution until the granulation point to form a meloxicam granule;
and the second granulated material is obtained by
preparing a second granulation solution comprising a mixture of a pharmaceutically acceptable alcohol and water;
manufacturing a second granulation support comprising an agglutinant agent and methocarbamol; and
moisturizing the second granulation support with the second granulation solution until the granulation point to form a methocarbamol granule; wherein
the meloxicam granule and the methocarbamol granule are optionally mixed with a lubricant.

5. (canceled)

6. The pharmaceutical composition of claim 1, wherein the pharmaceutical composition is useful for the treatment of disorders associated with muscle and problems associated with moderate to severe pain.

7. (canceled)
